(19) United States
(12) Patent Application Publication
(10) Pub. No.: US 2004/0213857 A1
(43) Pub. Date: Oct. 28, 2004

(54) Multi-vitamin and mineral supplement for pregnant women

(75) Inventors: Fabio Soldati, Savosa (CH); Lara Ambrosetti-Giudici, Malvaglia,Ticino (CH)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/825,477

(22) Filed: Apr. 15, 2004

Related U.S. Application Data
(60) Provisional application No. 60/499,429, filed on Sep. 2, 2003.

(30) Foreign Application Priority Data
Apr. 17, 2003 (EP) EP 03 008 985.8

(51) Int. Cl. A61K 33/32; A61K 33/04; A61K 33/34

(52) U.S. Cl. 424/638; 424/646; 424/643; 424/702; 424/682; 514/168; 514/251; 514/458; 514/474; 514/350; 514/356; 514/52; 514/393

(57) ABSTRACT

The invention relates to pharmaceutical or dietary unit dosage form, which can be swallowed easily, consisting essentially of vitamins, minerals and trace elements recommended for consumption by pregnant women, lactating women or women of childbearing potential that are attempting to become pregnant, DHA and a pharmaceutically or dietetically suitable carrier.

### RELATED APPLICATIONS

Benefit of U.S. Provisional Application Serial No. 60/499,429, filed on Sep. 2, 2003, is hereby claimed, and which application is incorporated herein in its entirety.

### BACKGROUND OF THE INVENTION

1. Technical Field

The invention relates to pharmaceutical or dietary unit dosage form, which can be swallowed easily, consisting essentially of vitamins and minerals recommended for consumption by pregnant women, lactating women or women of childbearing potential that are attempting to become pregnant, DHA and a pharmaceutically or dietetically suitable carrier.

2. Background Information

Vitamin and mineral preparations are commonly administered to treat specific medical conditions or as general nutritional supplements. Recent studies have elucidated the important physiological roles played by vitamins and minerals, and established a correlation between deficiencies or excesses of these nutrients and the etiologies of certain disease states in humans. See, e.g., Diplock, “Antioxidant Nutrients and Disease Prevention: An Overview,” Am. J. Clin. Nutr., 53:189-193 (1991); Documenta Geigy Scientific Tables, 457-497, (Diem and Cemtuer eds., 7th ed., 1975).

It has further become recognized that various, groups of the human population require different quantities and types of vitamins and minerals to prevent or alleviate diseases, as well as to maintain general good health. For example, it is known that pregnant women commonly require iron therapy to prevent or treat iron-deficiency anemia. Various prior patents have been directed to improving the efficacy of iron supplements for use during pregnancy. U.S. Pat. No. 4,994,283, for example, discloses nutritional mineral supplements which include iron and calcium compounds in combination with citrates or tartrates, ascorbates, and fructose. The tendency of calcium to inhibit the bioavailability of iron is said to be reduced in such compositions, so that the conjoint bioavailability of these two minerals is enhanced.

U.S. Pat. No. 4,431,634 maximizes the bioavailability of iron in prenatal iron supplements by maintaining the amount of calcium compounds in the supplement at 300 mg or less and the amount of magnesium compounds at 75 mg or less per dosage unit.

Another approach to the same problem is found in U.S. Pat. No. 4,752,479, wherein a multi-vitamin and mineral dietary supplement is provided which includes (a) one or more divalent dietary mineral components such as calcium or magnesium; and (b) a bioavailable iron component, present in a controlled release form and adapted to be released in a controlled manner in the gastrointestinal tract.

U.S. Pat. No. 4,710,387 discloses a nutritional supplement preparation for pregnant and breast-feeding women which contains 10-20% by weight of protein, 16-28% by weight of fat, 43-65% by weight carbohydrates, and at most 3.5% by weight of moisture, minerals, trace elements and vitamins. There is no provision for the use DHA or to easily swallowed nature of the capsules or tablets.

The International Patent Application WO 99/53777 discloses a nutritional composition in form of water based drinks or cereal bars for pregnant and lactating women comprising a non-milk protein source, a dietary fiber, a source of polyunsaturated fatty acids including DHA, minerals and vitamins. There is no hint to of a capsules or tablets, beads or lozenges.

The International Patent Application WO 00/66133 discloses a nutritional composition for administration before and during pregnancy comprising certain amounts of Vitamin B6, folic acid, magnesium and optionally calcium and DHA. Moreover, all the compositions disclosed therein contain additionally high amounts of calcium, resulting in a comparable voluminous dosage form, which is hard to swallow.

The International Patent Application WO 01/87070 discloses a nutritional supplement comprising vitamins minerals and a fish oil granulate in pulverized form comprising eicosapentenoic acid (EPA) and DHA. Moreover, all the compositions disclosed therein contain high amounts of calcium, resulting in comparable voluminous dosage form of 1000 mg or more containing about 20 mg DHA, which is hard to swallow and contains about 2% by weight of DHA only.

The U.S. patent application US 20020102330 A1 provides food bars for consumption by pregnant women containing one or more vitamins and/or minerals, and one or more anti-constipation and regularity-maintaining agents, which in addition may contain DHA.

The U.S. patent application US 20030050341 A1 suggests a composition for supplementing the diet comprising more than 100 mg of DHA. However, there is no hint to the trace elements Chromium, Copper, Molybdenum and Selenium. Moreover, all the compositions disclosed therein contain additionally high amounts of calcium, resulting in a comparable voluminous dosage form, which is hard to swallow.

Despite the foregoing efforts to improve vitamin and mineral supplementation for pregnant women, conventional prenatal supplements exhibit several deficiencies. One notable problem is that due to the high amount of calcium and the comparably high amount of vitamins the dosage form becomes very voluminous and hard to swallow especially for pregnant women.

Morning sickness generally causes a loss of appetite and a feeling of nausea, and is experienced by a significant number of pregnant women. Because they experience morning sickness, and because the pills and/or food bars that contain a high dose of recommended prenatal vitamins and minerals generally are very large in size, many pregnant women are often reluctant to take their prenatal vitamin and mineral pills or food bars. Further, when they do take these pills or food bars, these pregnant women often experience difficulty swallowing and retaining them. Problems, thus, arise concerning patient compliance (the daily consumption of vitamin and mineral supplements), maintaining or enhancing the health of pregnant woman, and the absorption of the quantity of vitamins and minerals that are associated with proper fetal development.

Moreover, during morning sickness pregnant women prefer to swallow capsules, pills or tablets than eating a food bar. The intake of a food bar requires that the bar is chewed by the woman; chewing the bar women feel the taste and this will usually have a negative impact on the nausea originated by the morning sickness.

Morning sickness generally occurs most frequently during the first trimester of pregnancy. Defects in the neural tube of a developing fetus (spina bifida) can also occur during the first trimester of pregnancy, for example, during the first month of gestation, before a woman may have become aware of her pregnancy. These defects are known to be linked to an inadequate intake of folic acid. It is well known that folic acid prevents neural tube defects. Thus, folic acid should be consumed in sufficient quantities by women of child-bearing ages. Folic acid has also been shown to have beneficial cardiac effects, and to decrease the risk of cervical dysplasia.

Moreover, the vitamin and mineral preparations available up to now for pregnant women do not provide any active ingredient to improve foetus' healthy brain development and eye sight.

It would therefore be desirable to provide a prenatal multi-vitamin and mineral supplement which overcomes the aforementioned deficiencies of the prior art.

### BRIEF SUMMARY OF THE INVENTION

It has now surprisingly been found that a pharmaceutical or dietary composition in form of a capsule or tablet, bead or lozenge, which can be swallowed easily, consisting essentially of (a) one or more vitamins, (b) one or more minerals selected from the group consisting of Iron, Zinc and Magnesium, (c) one or more trace elements selected from the group consisting of Chromium, Copper, Iodine, Molybdenum and Selenium, (d) DHA, and (e) a pharmaceutically or dietetically suitable carrier does overcome the above mentioned disadvantages of the known multi-vitamin and mineral supplements for pregnant women.

Accordingly the invention relates to a pharmaceutical or dietary composition in form of a capsule or tablet, bead or lozenge, which can be swallowed easily, consisting essentially of (a) one or more vitamins, (b) one or more minerals selected from the group consisting of Iron, Zinc and Magnesium, (c) one or more trace elements selected from the group consisting of Chromium, Copper, Iodine, Molybdenum and Selenium, (d) DHA, and (e) a pharmaceutically or dietetically suitable carrier.

Furthermore, the invention relates to a method of supplementing the dietary needs of a pregnant woman, a lactating woman or a woman of childbearing potential who is attempting to become pregnant, said method comprising administering to the woman a dietary supplementing amount of such a pharmaceutical or dietary composition.

Moreover, the invention relates to use of such a pharmaceutical or dietary composition, for the preparation of a pharmaceutical or dietary composition for supplementing the dietary needs of a pregnant woman, a lactating woman or a woman of childbearing potential who is attempting to become pregnant.

### DETAILED DESCRIPTION OF THE INVENTION

The present invention may be understood more readily by reference to the following detailed description of the preferred embodiments of the invention, and to the example included therein.

The term “pharmaceutical composition” means a composition, which is suitable for prescription and OTC medicaments, and which are available from doctors, in chemist's shop or in drugstores, only.

The term “dietary composition” means a composition, which is for supplementing the regular food intake with additional nutritional elements to enhance quality of life, and which are freely available without prescription in groceries or super market, but not only in drugstores.

The pharmaceutical or dietary composition is formulated in the form of capsules, tablets, beads or lozenges, preferably as soft shell capsules or tablets.

Pre-selected amounts of the composition of the present invention containing vitamin(s) (a), minerals (b), trace elements (c), and DHA(d) are preferably encapsulated in a soft gelatin including bovine, porcine, vegetable and succinylated gelatin shell. Optionally, the soft gelatin shell is essentially transparent so as to enhance the aesthetic qualities of the capsule. The soft gelatin shells as a rule comprise the following essential, as well as optional, components.

Gelatin is an essential component of the soft gelatin shells of the instant invention. The starting gelatin material used in the manufacture of soft capsules is obtained by the partial hydrolysis of collagenous material, such as the skin, white connective tissues, or bones of animals. Gelatin material can be classified as Type A gelatin, which is obtained from the acid-processing of porcine skins and exhibits an iso-electric point between pH 7 and pH 9; and Type B gelatin, which is obtained from the alkaline-processing of bone and animal (bovine) skins and exhibits an isoelectric point between pH 4.7 and pH 5.2. Blends of Type A and Type B gelatins can be used to obtain a gelatin with the requisite viscosity and bloom strength characteristics for capsule manufacture. Gelatin suitable for capsule manufacture is commercially available from the Sigma Chemical Company, St. Louis, Mo. For a general description of gelatin and gelatin-based capsules, see Remington's Pharmaceutical Sciences, 16th ed., Mack Publishing Company, Easton, Pa. (1980), page 1245 and pages 1576-1582; and U.S. Pat. No. 4,935,243, to Borkan et at., issued Jun. 19, 1990; these two references being incorporated herein by reference in their entirety.

The soft gelatin shell of the capsules of the instant invention, as initially prepared, comprises from about 20% to about 60% gelatin, more preferably from about 25% to about 50% gelatin, and most preferably from about 40% to about 50% gelatin. The gelatin can be of Type & Type B, or a mixture thereof with bloom numbers ranging from about 60 to about 300.

A plasticizer is another component of the soft gelatin shells of the instant invention. One or more plasticizers are incorporated to produce a soft gelatin shell. The soft gelatin thus obtained has the required flexibility characteristics for use as an encapsulation agent. Useful plasticizers of the present invention include glycerin, sorbitan, sorbitol, or similar low molecular weight polyols, and mixtures thereof.

The shell of the present invention, as initially prepared, generally comprises from about 10% to about 35% plasticizer, preferably from about 10% to about 25% plasticizer, and most preferably from about 10% to about 20% plasticizer. A preferred plasticizer useful in the present invention is glycerin.

The soft gelatin shells of the instant invention also comprise water. Without being limited by theory, the water is believed to aid in the rapid dissolution or rupture of the soft gelatin shell upon contact with the gastrointestinal fluids encountered in the body.

The shell of the present invention, as initially prepared, generally comprises from about 15% to about 50% water, more preferably from about 25% to about 40% water, and most preferably from about 30% to about 40% water.

Other optional components which can be incorporated into the soft gelatin shells include colorings including color coatings, flavorings, preservatives, anti-oxidants, essences, and other aesthetically pleasing components.

The compositions of the present invention can be encapsulated within any conventional soft gelatin shell that is capable of substantially containing the composition for a reasonable period of time. The soft gelatin shells of the instant invention can be prepared by combining appropriate amounts of gelatin, water, plasticizer, and any optional components in a suitable vessel and agitating and/or stirring while heating to about 65° C., until a uniform solution is obtained. This soft gelatin shell preparation can then be used for encapsulating the desired quantity of the fill composition employing standard encapsulation methodology to produce one-piece, hermetically-sealed, soft gelatin capsules. The gelatin capsules are formed into the desired shape and size so that they can be readily swallowed. The soft gelatin capsules of the instant invention are of a suitable size for easy swallowing and typically contain from about 100 mg to about 2000 mg of the active composition. Soft gelatin capsules and encapsulation methods are described in P. K. Wilkinson et at., “Softgels: Manufacturing Considerations”, Drugs and the Pharmaceutical Sciences, 41 (Specialized Drug Delivery Systems), P. Tyle, Ed. (Marcel Dekker, Inc., New York, 1990) pp.409-449; F. S. Horn et at., “Capsules, Soft”, Encyclopedia of Pharmaceutical Technology, vol. 2, J. Swarbrick and J. C. Boylan, eds. (Marcel Dekker, Inc., New York, 1990) pp. 269-284; M. S. Patel et at., “Advances in Softgel Formulation Technology”, Manufacturing Chemist, vol. 60, no. 7, pp. 26-28 (July 1989); M. S. Patel et al., “Softgel Technology”, Manufacturing Chemist, vol. 60, no. 8, pp. 47-49 (August 1989); R. F. Jimerson, “Softgel (Soft Gelatin Capsule) Update”, Drug Development and Industrial Pharmacy (Interphex '86 Conference), vol. 12, no. 8 & 9, pp. 1133-1144 (1986); and W. R. Ebert, “Soft Elastic Gelatin Capsules: A Unique Dosage Form”, Pharmaceutical Technology, vol. 1, no.5, pp. 44-50 (1977); these references are incorporated by reference herein in their entirety. The resulting soft gelatin capsule is soluble in water and in gastrointestinal fluids. Upon swallowing the capsule, the gelatin shell rapidly dissolves or ruptures in the gastrointestinal tract thereby introducing the pharmaceutical actives from the liquid core into the physiological system.

Preferably the capsules have an oblong shape to facilitate swallowing. In the case of a capsule containing 300 to 700 mg of the combined active ingredients an oblong capsule may be about 10-28 mm, preferably 20-26 mm, in particular about 25 mm long and have a diameter of about 5 to 11 mm, preferably 6-10 mm, in particular 8-9 mm.

Tablets of the invention will generally contain at least one pharmaceutically or dietary acceptable excipient conventionally used in the art of solid dosage form formulation. Suitable excipients which may be incorporated include lubricants, for example magnesium stearate and stearic acid; disintegrants, for example cellulose derivatives; starches; binders, for example modified starches, polyvinylpyrrolidone and cellulose derivatives; glidants, for example colloidal silicas; compression aids, for example cellulose derivatives; as well as preservatives, suspending agents, wetting agents, flavoring agents, bulking agents, adhesives, coloring agents, sweetening agents appropriate to their form.

Suitably when the composition is in a tablet form, the composition will further comprise a film coat, e. g. hydroxypropylmethylcellulose (HPMC). Suitably the film coat is a transparent film coat, although an opaque film coat e. g. as obtained when using a film coat material in combination with an opacifier or a pigment such as titanium dioxide, a lake or a dye, may also be used. Advantageously it has been found that the inclusion of an opaque film coat minimizes tablet discoloration, which may occur on long-term storage of the tablet. Discoloration may also be avoided by incorporating a coloring agent into the tablet core. Suitably such tablets may also be film-coated, e. g. if desired for aesthetic purposes and/or to aid swallowing.

The combined active ingredients are mixed with the excipients of the tablet core and compressed on a suitable tablet press.

The compression forces which are needed to produce tablets of suitable breaking resistance and hence with the required breakdown times are dependent on the shapes and sizes of the punching tools used. Compression forces in the range from 2-20 kN are preferred. Higher compression forces may lead to tablets with a delayed released of the active substances (i) to (iv). Lower compression forces may produce mechanically unstable tablets. The tablet cores may have different shapes; the preferred shapes are round biplanar or biconvex and oval or oblong forms.

The coating solution is prepared by mixing the film-forming agent with the colouring materials and a plasticizer in water. Using a suitable coating pan the film-coating solution is applied on to the tablet cores.

Preferably the tablets have an oblong shape to facilitate swallowing. In the case of a film-coated tablet containing 300 to 700 mg of the combined active ingredients an oblong tablet may be about 10-20 mm long and have a width of about 5 to 10 mm.

As a rule the tablets according to the present invention contain lower amounts of DHA (d) than the capsules due to the oily nature of DHA.

A wide variety of vitamins, minerals and trace elements that are safe for consumption by pregnant women, lactating women or women having childbearing potential that are attempting to become pregnant may be used in the dosage form of the invention in varying quantities. These vitamins, minerals and trace elements include, for example, Vitamin A or beta-carotene, Vitamin B₁ (as Thiamin or Thiamin mononitrate), Vitamin B₂ (as Riboflavin), Vitamin B₃ (as Niacin), Vitamin B₆ (as Pyridoxine or Pyridoxine hydrochloride), Vitamin B₉ (Folic Acid), Vitamin B₁₂ (cyanocobalamine), Vitamin H (Biotin), Vitamin C (Ascorbic Acid), Vitamin D, Vitamin E (as d1-Alpha Tocopherol Acetate), Vitamin K, Folacin, Niacinamide, Iron (as Ferrous Fumarate), Phosphorus, Pantothenic Acid (as Calcium Pantothenate), Iodine (as Potassium Iodide), Magnesium (as Magnesium Oxide), Zinc (as Zinc Oxide), Selenium (as Sodium Selenate), Copper (as Cupric Oxide), Manganese (as Manganese Sulfate), Chromium (as Chromium Chloride), Molybdenum (as Sodium Molybdate), Choline, Fluoride, Chloride, Potassium, Sodium, and mixtures thereof. Such vitamins, minerals and trace elements are commercially available from sources known by those of skill in the art, such as Hoffmann-LaRoche Inc. (Nutley, N.J.).

Preferably the composition according to the invention contains at least one vitamin selected from the group consisting of β-carotene, Vitamin B₁, Vitamin B₂, Vitamin B₆, Vitamin B₁₂, Vitamin C, Vitamin D₃, Vitamin E, Folic Acid, Biotin and Niacinamide, in particular such dosage forms, in which the multivitamin mixture consists of β-carotene, Vitamin B₁, Vitamin B₂, Vitamin B₆, Vitamin B₁₂, Vitamin C, Vitamin D₃, Vitamin E, Folic Acid, Biotin and Niacinamide.

Preferably, the weight ratio of DHA (d) to at least one of the vitamins (a) selected from the group consisting of Vitamin D3 and Biotin is from 500:1 to 100,000:1, preferably 3,000:1 to 30,000:1.

Furthermore preferred is a composition, wherein the weight ratio of folic acid to Vitamin B6 is from 1:1 to 1:8, preferably 1:1.5 to 1:7.5, in particular about 1:3.

Furthermore preferred is a composition, wherein weight ratio of Iron to Magnesium is from 10:1 to 1 to 2, in particular from 5:1 to 1:1, most preferred about 2.7:1.

The composition according to the invention contains at least two minerals selected from the group consisting of Iron, Zinc and Magnesium, and at least two trace elements selected from Chromium, Copper, Iodine, Molybdenum and Selenium, preferred are composition comprising at least Molybdenum and/or Selenium, in particular such compositions, in which the mixture of minerals and trace elements consists of Iron, Zinc, Magnesium, Chromium, Copper, Iodine, Molybdenum and Selenium.

Pre-mixes containing vitamins and minerals recommended for pregnant women, lactating women and women having childbearing potential that are attempting to become pregnant that may be employed to produce the unit dosage form of the present invention may be obtained from Watson Foods Co., Inc. under Watson Code WT-6061A.

The dosage forms of the invention may be formulated using any pharmaceutically-acceptable forms of the vitamins and/or minerals described above, including their salts, which are known by those of skill in the art. For example, useful pharmaceutically-acceptable magnesium compounds include Magnesium Stearate, Magnesium Carbonate, Magnesium Oxide, Magnesium Hydroxide and Magnesium Sulfate. Pharmaceutically-acceptable iron compounds include any of the well-known Iron II (ferrous) or Iron III (ferric) supplements, such as Ferrous Sulfate, Ferric Chloride, Ferrous Gluconate, Ferrous Lactate, Ferrous Tartrate, Iron-Sugar-Carboxylate complexes, Ferrous Fumarate, Ferrous Succinate, Ferrous Glutamate, Ferrous Citrate, Ferrous Pyrophosphate, Ferrous Cholinisocitrate, Ferrous Carbonate, and the like.

The vitamins and/or minerals used to prepare the dosage forms of the invention may be microencapsulated in a coating of fat, microcrystalline cellulose or similar material in order to prevent their degradation under various conditions.

The vitamins and/or minerals that are employed in the dosage form of the invention are those that are recommended for consumption by pregnant women, lactating women or women having childbearing potential that are attempting to become pregnant. These vitamins and minerals are employed in an amount that is effective for enhancing the nutrition of pregnant women, lactating women or women having childbearing potential that are attempting to become pregnant, or of their developing fetuses or babies. This quantity will vary depending upon the particular vitamins and/or minerals chosen for use, but generally ranges from about 25 to about 95 weight percent of the total weight of the dosage form, and preferably ranges from about 30 to about 90 weight percent, with about 80 weight percent being most preferred.

Each dosage form may contain one or more of the above vitamins, minerals and/or trace elements in any quantity that is safe for consumption by pregnant women, lactating women or women having childbearing potential that are attempting to become pregnant (i.e., a quantity that would not cause harm to the woman consuming the food bar, or to her developing fetus or breast-feeding baby). Set forth herein below are the approximate preferred ranges of the daily quantities of the various vitamins and minerals that may generally be used in one dosage form (or divided between more than one dosage form for consumption during a one-day period) for pregnant women, lactating women or women having childbearing potential that are attempting to become pregnant (from about one quantity to about another quantity), as well as more preferred ranges, and the most preferred quantities.

Most preferred each dosage form according to the present invention contains one or more of the above vitamins, minerals and/or trace elements in an amount which corresponds to about 100% of the recommended daily dose for pregnant women.

DHA (docosahexaenoic acid) is a long-chain fatty acid that is necessary for brain and eye development in children, and is included as an ingredient of the dosage form of the invention in an amount ranging from about 10 to about 300 mg, with about 100 to 200 mg being preferred, and about 150 mg being most preferred for pregnant women, lactating women, and women having childbearing potential that are attempting to become pregnant.

A wide variety of fats and oils can be employed as carriers of DHA (d). These fats or oils include, for example, olive oil, canola oil, palm oil, coconut oil, sunflower oil, peanut oil, vegetable oil, lecithin, fish oil, cotton seed oil, soybean oil, lard, monoglycerides, diglycerides, butter, margarine, and other animal, vegetable, and marine fats, and milk fats, waxes such as beeswax, which are commercially available from sources known by those of skill in the art, and mixtures thereof. Vegetable oil is the preferred fat for use in the food bars of the invention.

Particularly preferred are dosage forms according to the invention consisting essentially of

(a) a multi-vitamin mixture consisting of β-carotene, Vitamin B₁, Vitamin B₂, Vitamin B₆, Vitamin B₁₂, Vitamin C, Vitamin D₃, Vitamin E, Folic Acid, Biotin and Niacinamide;

(b) a mineral mixture consisting of Iron, Zinc and Magnesium;

(c) a mixture of trace elements consisting of Chromium, Copper, Iodine, Molybdenum and Selenium;

(d) DHA; and

(e) a pharmaceutically or dietetically suitable carrier.

More preferred is a dosage form according to the invention consisting essentially of

(a) 100 to 160 mg of a multi-vitamin mixture consisting of β-carotene, Vitamin B₁, Vitamin B₂, Vitamin B₆, Vitamin B₁₂, Vitamin C, Vitamin D₃, Vitamin E, Folic Acid, Biotin and Niacinamide;

(b) 60 to 120 mg a mineral mixture consisting of Iron, Zinc and Magnesium;

(c) 100 to 5000 μg a mixture of trace elements consisting of Chromium, Copper, Iodine, Molybdenum and Selenium;

(d) 100 to 200 mg of DHA; and

(e) a pharmaceutically or dietetically suitable carrier.

Most preferred is a dosage form according to the invention consisting essentially of

(a) a multi-vitamin mixture consisting of 1.5 to 2.5 mg of β-carotene, 1.0 to 1.8 of mg Vitamin B₁, 1.0 to 1.8 mg of Vitamin B₂, 1.5 to 2.5 mg of Vitamin B₆, 1.0 to 5.0 μg of Vitamin B₁₂ₘ , 60 to 110 mg of Vitamin C, 2.0 to 200 μg, in particular 2.5 to 10 μg of Vitamin D₃, 15 to 30 mg of Vitamin E, 200 to 1000 μg of Folic Acid, 10 to 100 μg of Biotin and 10 to 40 mg of Niacinamide;

(b) a mineral mixture consisting of 10 to 50 mg of Iron, 5 to 20 mg of Zinc and 1 to 100 mg of Magnesium;

(c) a mixture trace elements consisting of 10 to 50 μg of Chromium, 0.5 to 1.5 mg of Copper, 50 to 500 μg of Iodine, 10 to 100 μg of Molybdenum and 10 to 100 μg of Selenium;

(d) 100 to 200 mg, in particular about 150 mg of DHA; and

(e) a pharmaceutically or dietetically suitable carrier.

Most preferably the weight of the active ingredients (a) to (d) in the unit dosage form of the composition according to the invention is from 150 to 700 mg, in particular from 200 to 600 mg, most preferably from about 300 to 400 mg.

One unit dosage form of the composition according to the invention is preferably administered per day.

Procedures by way of example for preparing the dosage form according to the invention will be described in more detail hereinafter. The example which follows serves solely as a detailed illustration without restricting the subject matter of the invention.

### EXAMPLE 1

### Soft Capsules

Soft gelatine capsules are prepared containing the following active ingredients:

| Components | Effective amount/caps. | |
|---|---|---|
| Active ingredients | | |
| β-Carotene | 2.8 | mg |
| as 30% suspension | 9.338 | mg |
| Thiamine mononitrate (Vitamin B1) | 1.75 | mg |
| Riboflavin (Vitamin B2) | 1.68 | mg |
| Pyridoxine hydrochloride (Vitamin B6) | 2.09 | mg |
| Cyanocobalamin | 3.25 | μg |
| as Cyanocobalamin 0.1% with Mannitol | 3.25 | mg |
| Ascorbic acid (Vitamin C) | 114.75 | mg |
| Cholecalciferol (Vitamin D3) | 5.75 | μg (230 IU) |
| d,l-α-Tocopherol acetate (Vitamin E) | 24.59 | mg |
| Folic acid | 720 | μg |
| Biotin | 31.5 | μg |
| Nicotinamide (Niacin, Vitamin PP) | 18.9 | mg |
| Chromium | 30 | μg |
| as Chromium chloride hexahydrate | 153.6 | μg |
| Copper | 1000 | μg |
| as Copper (II sulphate), dried | 2512 | μg |
| Iron | 27.0 | mg |
| as Ironfumarate | 82.14 | mg |
| Iodine | 200 | μg |
| as Potassium Iodide | 261.6 | μg |
| Molybdenum | 50 | μg |
| as Sodium molybdate dihydrate | 126.1 | μg |
| Selenium | 60 | μg |
| as Sodium selenite dried | 133.2 | μg |
| Zinc | 11 | mg |
| as Zinc sulphate monohydrate | 30.25 | mg |
| Magnesium | 10 | mg |
| as Magnesium sulphate dried | 71.0 | mg |
| DHA | 150 | mg |
| as DHA Oil 50% | 300 | mg |

The ingredients are mixed and encapsulated into gelatin, water and a plasticizer to form oblong soft gelatin capsules having the following dimensions:

Diameter: 7 to 11, preferably 8 to 9 mm;

Length: 21 to 26, preferably about 25 mm.

1. A pharmaceutical or dietary composition consisting essentially of (a) one or more vitamins, (b) one or more minerals, (c) one or more trace elements, (d) DHA, and (e) a pharmaceutically or dieterically suitable carrier.

2. A pharmaceutical or dietary composition according to claim 1, wherein one or more vitamins is selected from the group consisting of β-carotene, Vitamin B₁, Vitamin B₂, Vitamin B₆, Vitamin B₁₂, Vitamin C, Vitamin D₃, Vitamin E, Folic Acid, Biotin and Niacinamide.

3. A pharmaceutical or dietary composition according to claim 1, wherein one or more trace elements is selected from the group consisting of Chromium, Copper, Iodine Molybdenum and Selenium.

4. A pharmaceutical or dietary composition according to claim 1, wherein one or more minerals is selected from the group consisting of Iron, Zinc and Magnesium.

5. A pharmaceutical or dietary composition according to claim 1, wherein the weight ratio of DHA to at least one of the vitamins selected from Vitamin D₃ and Biotin is from about 500:1 to about 100,000:1

6. A pharmaceutical or dietary composition according to claim 2, wherein the weight ratio of folic acid to Vitamin B₆ is from about 1:1 to about 1:8.

7. A pharmaceutical or dietary composition according to claim 1, wherein the weight ratio of DHA to at least one trace element selected from Chromium, Copper, Iodine Molybdenum and Selenium is from about 500:1 to about 20,000:1.

8. A pharmaceutical or dietary composition according to claim 4, wherein the weight ratio of Iron to Magnesium is from about 10:1 to about 1:2.

9. A pharmaceutical or dietary composition according to claim 1 which is in the form of a capsule, tablet, bead or lozenge.

10. A pharmaceutical or dietary composition consisting essentially of
(a) a multi-vitamin mixture consisting of β-carotene, Vitamin B₁, Vitamin B₂, Vitamin B₆, Vitamin B₁₂, Vitamin C, Vitamin D₃, Vitamin E, Folic Acid, Biotin and Niacinamide;
(b) a mineral mixture consisting of Iron, Zinc and Magnesium;
(c) a mixture of trace elements consisting of Chromium, Copper, Iodine, Molybdenum and Selenium;
(d) DHA; and
(e) a pharmaceutically or dietetically suitable carrier.

11. A pharmaceutical or dietary composition according to claim 10, wherein the weight of the active ingredients (a) to (d) is in unit dosage form is from about 150 to about 700 mg.

12. A pharmaceutical or dietary composition according claim 10 consisting essentially of
(a) from about 100 to about 160 mg of a multi-vitamin mixture consisting of β-carotene, Vitamin B₁, Vitamin B₂, Vitamin B₆, Vitamin B₁₂, Vitamin C, Vitamin D₃, Vitamin E, Folic Acid, Biotin and Niacinamide;
(b) from about 60 to about 120 mg of a mineral mixture consisting of Iron, Zinc and Magnesium;
(c) from about 100 to about 5000 μg of a mixture of trace elements consisting of Chromium, Copper, Iodine, Selenium and Molybdenum;
(d) from about 100 to about 200 mg of DHA; and
(e) a pharmaceutically or dietetically suitable carrier.

13. A pharmaceutical or dietary composition according claim 10 consisting essentially of
(a) a multi-vitamin mixture consisting of 1.5 to 3.5 mg of β-carotene, 1.0 to 1.8 of mg Vitamin B₁, 1.0 to 1.8 mg of Vitamin B₂, 1.5 to 2.5 mg of Vitamin B₆, 1.0 to 5.0 μg of Vitamin B₁₂, 60 to 150 mg of Vitamin C, 2.0 to 200 μg of Vitamin D₃, 15 to 30 mg of Vitamin E, 200 to 1000 μg of Folic Acid, 10 to 100 μg of Biotin and 10 to 40 mg of Niacinamide;
(b) a mineral mixture consisting of 10 to 50 mg of Iron, 5 to 20 mg of Zinc and 1 to 100 mg of Magnesium;
(b) a mineral mixture consisting of 10 to 50 μg of Chromium, 0.5 to 1.5 mg of Copper, 50 to 500 μg of Iodine, 10 to 100 μg of Molybdenum and 10 to 100 μg of Selenium;
(d) 100 to 200 mg of DHA; and
(e) a pharmaceutically or dietetically suitable carrier.

14. A pharmaceutical or dietary composition according to claim 10 which is in the form of an oblong gelatine capsule having the following dimensions:
diameter: 7 to 11 mm; and
length: 21 to 26 mm.

15. A method of supplementing the dietary needs of a pregnant woman, a lactating woman or a woman of childbearing potential who is attempting to become pregnant, said method comprising administering to the woman in need thereof, a dietary supplementing effective amount of a pharmaceutical or dietary composition according to claim 1.

16. A method of supplementing the dietary needs of a pregnant woman, a lactating woman or a woman of childbearing potential who is attempting to become pregnant, said method comprising administering to the woman in need thereof, a dietary supplementing effective amount of a pharmaceutical or dietary composition according to claim 10.

17. A method of supplementing the dietary needs of a pregnant woman, a lactating woman or a woman of childbearing potential who is attempting to become pregnant, said method comprising administering to the woman in need thereof, a dietary supplementing effective amount of a pharmaceutical or dietary composition according to claim 11.

18. A method of supplementing the dietary needs of a pregnant woman, a lactating woman or a woman of childbearing potential who is attempting to become pregnant, said method comprising administering to the woman in need thereof, a dietary supplementing effective amount of a pharmaceutical or dietary composition according to claim 12.

19. A method of supplementing the dietary needs of a pregnant woman, a lactating woman or a woman of childbearing potential who is attempting to become pregnant, said method comprising administering to the woman in need thereof, a dietary supplementing effective amount of a pharmaceutical or dietary composition according to claim 13.

20. A method of supplementing the dietary needs of a pregnant woman, a lactating woman or a woman of childbearing potential who is attempting to become pregnant, said method comprising administering to the woman in need thereof, a dietary supplementing effective amount of a pharmaceutical or dietary composition according to claim 14.
